(19) United States
(12) Patent Application Publication
(10) Pub. No.: US 2003/0022223 A1
(43) Pub. Date: Jan. 30, 2003

(54) Methods for scoring single nucleotide polymorphisms

(76) Inventor: Rebecca Rone, Burlington, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/194,146

(22) Filed: Jul. 12, 2002

Related U.S. Application Data
(60) Provisional application No. 60/305,042, filed on Jul. 12, 2001.

(51) Int. Cl. C12Q 1/68; G06F 19/00; G01N 33/48; G01N 33/50

(52) U.S. Cl. 435/6; 702/20

(57) ABSTRACT

The invention provides a method for the probabilistic scoring of SNPs for prioritization of target genes and proteins. The invention includes a method for identifying and evaluating a polynucleotide sequence encoding a polypeptide or a non-coding sequence to predict a correlation with a pathological state. A probabilistic scoring mechanism is used for the evaluation of the biological significance of SNPs and the use of this score for the prioritization of target genes and proteins.

This application claims priority to U.S. Provisional Patent Application Serial No. 60/305,042, filed on July 12, 2001.

### BACKGROUND OF THE INVENTION

This invention relates to DNA analysis.

Single nucleotide polymorphisms (SNPs) are DNA sequence variations that occur when a single nucleotide (A,T,C, or G) in a gene sequence is changed. SNPs can occur every 100 to 300 bases along the human genome and may be associated with a disease state. For example, a SNP at a certain genetic location may indicate a pathological state or a predisposition to develop a pathology. SNPs are generally stable and can be followed from generation to generation.

### SUMMARY OF THE INVENTION

The invention provides a method for the probabilistic scoring of SNPs for prioritization of target genes and proteins. The invention includes a method for identifying and evaluating a polynucleotide sequence encoding a polypeptide or a non-coding sequence to predict a correlation with a pathological state. A probabilistic scoring mechanism is used for the evaluation of the biological significance of SNPs and the use of this score for the prioritization of target genes and proteins. The central concept is to combine measurements of mutational diversity in use for bioinformatics with measurements of protein structure in use for evaluating and modeling proteins. These measurements are used directly (e.g., in an additive fashion) to create the scoring. Alternatively, the measurements are combined using neural nets or genetic algorithms or similar mathematical constructs. The implementation of the method results in the creation of a software package that can formulate a QSAR-SNP (Quantitative Structure Activity Relationship based on Single Nucleotide Polymorphism) equation or a probabilistic score for the SNP.

The method is based on detecting a proline conversion in a gene. A proline conversion is a genetic polymorphism that leads to an insertion or substitution of a proline residue in an amino sequence or a polymorphism that leads to an elimination of a proline residue in an amino acid sequence. A proline conversion is indicative of disease associated SNP, because a gain or loss of a proline residue in a protein affects the secondary and tertiary structure of the protein (which, in turn, affects the function of the protein). For example, prolines are often located at the end of an alpha helix structure in a protein (and serve to terminate such a structure). Loss of a proline in such a position leads to a protein structure in which the alpha helix terminates at a position which differs from that of the native wild type protein. Similarly, insertion of substitution of a proline residue in a region of a protein alters its structure such that a ligand binding site or other functional structure is disrupted (an event that leads to impaired biological function or loss of biological function) and development of a pathological state.

The DNA segments containing these highly predictive SNPs (whether known genes or not) are then presumed to be those that are most likely to be causative of disease rather than just effected by the disease state. These segments indicate a preferred target for drug discovery or further functional characterization. This step of target prioritization distinguishes target identification and target validation. The methods are used as a filter to focus the expenditure of resources on the more critical components of disease.

Gene sequences are analyzed by examining 5-residue windows (e.g., 5 nucleotide triplets) to identify a proline conversion. For example, if the SNP is at a given position, the sequences located 2 triplets upstream (I−2) and 2 triplets downstream (I+2) are examined to determine a proline conversion. A “CCX” (where X is any residue) triplet is not scored because it still encodes a proline residue (regardless of the identity of the third position in the triplet). A priority score or probabalistic score is expressed as follows: 0=no proline conversion, 1=proline conversion. The scores are added and optionally correlated with a genetic matrix to derive coefficient for a normalized score. The scores or coefficients are added to yield a total probabalistic score.

Accordingly, the method of identifying a disease-associated gene is carried out by obtaining first profiles of genes that are free of SNPs and corresponding second profiles of the genes containing at least one SNP; for each SNP residue in the second profiles, analyzing indicia of triplets of residues in the second profiles containing the SNP residue and corresponding triplets in the first profiles to determine whether any of the corresponding triplets indicate a proline conversion between the first and second profiles; and adding indicia of proline conversions for each of the genes to determine probability scores to identify a disease-associated gene. The method optionally includes a step of altering the probability scores in accordance with ontological information associated with the genes.

The invention also includes a chromosome profile containing a catalog of SNPs in which a SNP is prioritized with a probabalistic score (or sum of scores) as well as a gene profile containing catalog of SNPs in which a SNP is prioritized with a probabalistic score (e.g., 0 or 1).

Since the measurements currently in use are being used across the industry to uncover genes that share similar functions and proteins, which belong to similar families, then scoring of SNPs according to the invention yields superior results in situations in which current search mechanisms return so many similarities that the prioritization is required for efficiency and economy.

Other features, objects, and advantages of the invention will be apparent from the description and from the claims.

### DETAILED DESCRIPTION

The post-genomics era may become the era of understanding disease by understanding individual genotypes and their subpopulations, particularly single nucleotide polymorphisms called SNPs. An individual's genotype, including all their SNP's may be embedded on an electronic chip as is used currently for ATM cards. This card will be read electronically by one's doctor, who will obtain an immediate readout of possible drug reactions or suggested therapies to treat the individual's disease. The doctor may also be able to identify disease risk factors and assess their relative potential for harm to the individual. This is called personalized or individualized medicine. Tools may become available to assess what preventive life style changes or drug therapies are important for the individual. All references herein to “individual” are applicable to a subpopulation of individuals, which share a common or similar SNP distribution. The term DNA segment or genetic information does not preclude the conversion of all methods into protein code elements (amino acid residues). The methods are filly interchangeable between the two types of data which may be available (DNA code or protein code).

In many cases, it may be an over-simplification to expect disease states to be linked exclusively to single SNPs. Disease states with known mechanisms are often related not only to the protein mutations associated with a single SNP but other factors such as post-transcriptional modifications of mRNA, protein expression mechanisms, and post-translational modifications of protein. The result is that researchers are finding that SNPs are cross-correlated in complicated ways. Although proper annotation may eventually identify all of these cross-correlations it will probably take many years of laboratory research and bioinformatics technology application before this type of in-depth coverage is available for the majority of diseases. In the laboratory molecular biologists are currently discovering a huge number of proteins that are potential drug targets. In the current state of the science it becomes important to prioritize target proteins.

The invention features the use of a probablistic scoring algorithm using SNPs to characterize and prioritize putative target proteins. In this paradigm, SNPs that are most potentially damaging to protein structure and to mRNA expression pathways are presumed to form disease states, singly or in a cross-correlated haplotype fashion. This allows the focus to be a single gene. The effect of post-translational events is optionally incorporated into the evaluation. For example, the method is applied to the modeling of beta-adrenergic antagonists for asthma treatment as well as other agonists and antagonists for other disease states.

Another application for this algorithm is the creation of fingerprinting whereby the probabilistic scoring can be used to create profiles for the gene in question. These profiles are then used as markers to attempt to identify cross-correlated functions between genes. In this scenario the true predictive nature of the algorithm is less important than its ability to identify and track similarities in protein structure that may correlate to gene function.

Another problem facing industry in the post-genomics era, is that of scale. Studying one gene at a time may be characterized as an antiquated approach to studying disease. After all human genes have been properly identified and annotated, researchers will be able to concentrate on the variations, or polymorphisms, in the human genome with the aid of the described scoring methods. These variations or SNPs are thought to be the basis of disease. Understanding SNPs and the ways in which they are cross-correlated will play a major role in the growing science of predictive medicine and pharmacogenomics. In the post-genomics era, it will be important to move quickly from gene discovery to having gene products accessible on a technological platform for testing of potential drug compounds.

One critical interface for big pharmaceutical companies to address in order to hold their value is the biology-medicine interface. Bioinformatics can be applied to the growing area of pharmacogenomics, here defined as the genetic response of an individual or population to a drug, and SNPs, a single nucleotide polymorphism which may be silent or non-damaging to an organism or indicate a genetic mutation related to a disease or to drug response. Of course, not all disease states and/or drug responses are related to a single SNP, but to several SNPs along the pathway or to other factors, such as post-translational modifications.

Overall the investment in bioinformatics is low compared to the investment in clinical trials. But if bioinformatics can be leveraged in this area, it may help eliminate the drug exposure of subpopulations for which the drug candidate is non-efficacious or even life-threatening. One application of this field is the possibility of “rescue of drugs ”, whereby promising drug candidate compounds may be “resurrected ” in clinical trials if the subpopulation that are non-responders or negative responders can be identified. In this scenario, the subpopulation for which the drug is of real benefit can be clearly identified and used exclusively for population studies related to the drug's efficacy.

### Drug Selection for Patients

Bioinformatics can be used to track and correlate complicated biological information (1). For example, it is possible to select patients for optimal drug treatment strategies based on haplotypes. SNP information has been cross-correlated to predict responders to albuterol drug therapy for asthma. By generating, storing and analyzing expression information about the 13 individual SNP's found on the b2-adrenergic receptor gene, the researchers were able to identify a complicated relationship that exists between the individual SNPs and the haplotypes involved. By cross-correlating this information, it was found that of the possible 8,192 combinations, only 12 are actually found and only five occurred in the majority of patients. From this information, it is possible to predict which patients would benefit from the proposed treatment.

The 13 individual SNPs were discovered by applying bioinformatics software tools for haplotype analysis using: a) Clark's algorithm to assign haplotypes based on DNA sequence in normal homozygous individuals (2), b) phylogenetic analysis to find minimal spanning networks related to cross-correlations of the haplotypes (3), and c) quantification of linkage disequilibrium (4,5). Statistical analysis (6) was performed to locate haplotype pairings, which were diagnostic for treatment of asthma with albuterol. In their findings, a haplotype pair defined as 2/2 showed nearly 50% greater responsiveness than pair 4/4. Haplotype 2 is seen at varying frequencies in various racial groupings, whereas the frequency of haplotype 4 is similar among all groups. Another haplotype, 6, shows racial distinctions also and is presumed to be significant but the current data is not extensive enough to form a clear diagnostic marker. Haplotype 6 occurs frequently in African-Americans with asthma. Currently the high rate of asthma among African-Americans remains unexplained.

### Probabilistic Scoring Techniques

The technique described herein represents a probabilistic scoring mechanism for the evaluation of the biological significance of SNPs and the use of this score for the prioritization of target genes and proteins. The central concept is to combine measurements of mutational diversity in use for bioinformatics with measurements of protein structure in use for evaluating and modeling proteins. These measurements are used directly (e.g., in an additive fashion) to create the scoring or they can be combined using neural nets or genetic algorithms or similar mathematical constructs. A useful formulation of the method results in the creation of a QSAR-SNP (Quantitative Structure Activity Relationship based on Single Nucleotide Polymorphism).

The method is composed of one or more of the following components:

A neural net or genetic algorithm/both or neither

Measurements of mutational diversity

Measurements of protein structural diversity

Outcomes:

Measurements of disease state severity or other diagnostic markers

Measurements of drug therapy or other treatment response

In the techniques to be applied using the method outlined here, one begins with the following observation. Currently, a great deal is known about the nature of the effect of mutations on protein structure (25). In many cases, it appears that the mutations of a protein that create one organism versus another can be traced to a progressive change in the protein over eons of time. Even though it cannot be proven that this is the mechanism in use by mother nature, these correlations have been quantified in a series of programs in use in the bioinformatics industry. This is the basis of the PAM and other matrices used to measure diversity in proteins as a function of diversity in species. In corollary, there are also several matrices to describe protein structural elements based on single site mutations or on the mutations found in species diversity. These matrices are formulated to measure the stability or loss of stability in the structural integrity of the protein as a function of specific changes in the amino acid residue sequence, i.e. mutations.

The invention is based on the premise that the diversity in individuals which makes them susceptible to diseases or responsive to drug treatments or other therapies also makes their proteins diverse in subtle ways that are a reflection of the same processes in operation in diverse species. In other words, there is an entire spectrum of mutations that make human beings different from lower organisms. This spectrum is represented as a progressive series of SNPs becoming more and more numerous and diverse as the species become more and more diverse. In the case of diseased individuals, these SNPs are fewer and less diverse. These subtleties are manifested in their SNPs either as single genes or in the cross correlations that can be found. It is therefore possible to find these variations according to the invention, correlate them to their measured values as found in existing matrices based on species diversity or on changes in protein structure and then use the measured values to create an equation which can predict the importance of an individual SNP from the dataset in question. Individual genes that contain such SNPs are identified as a preferred target for investigation either in their genetic form or in their protein form. In the case of SNP modifications which effect the protein code (those having an identified open reading frame, ORF), then the preferred target is the protein form. In cases where a SNP is correlated to a disease or therapy but not contained in an ORF, then the preferred target is at first the genetic form, but this may optionally be reduced to the protein, which binds and uses this translational trigger. Ultimately when enough is understood about the entire genetic code all cases of targets should reduce to this protein form as the translational and post-translational events should be modulated by proteins that will finally be identified. The method described herein is used to implement or aid in this task as well as identify the underlying equations that demonstrate the cross correlations.

### Neural Nets and Genetic Algorithms

One difficulty arising from the use of a spectrum to describe SNP diversity is the fact that the diversity among individuals within a species is much more subtle than that between species. One way to address this problem is to use a neural net (NN) or genetic algorithm (GA) as the core of the method. In using a NN or GA, one can attach relative significance of the particular measurement under consideration and its correlation to the overall equation used to predict the importance of the SNP as seen in that dataset. An NN or GA when properly programmed to incorporate the measurements under question can automatically identify which measurements can be correlated with the SNP data in use and what coefficient needs to be applied to which measurement. The methods may reveal cases where the effects are so subtle as to be non-responsive to some measures and highly responsive to others. Therefore some SNPs will be highly predictive for a particular disease or several diseases and other SNPs will not.

As data is accumulated, it will be possible to infer that the SNPs showing the highest degree of correlation by this method are the proper targets for drug discovery whether contained in an ORF or not. From these data, not only individual QSAR-SNPs for a particular dataset and a particular disease are formulated, but also a combined database of equations that predict quantitatively the probabilistic determination that a particular SNP is the root cause of the disease and is therefore the proper target for drug discovery.

In order to obtain accurate predictions, not only the SNP in question but also the upstream and downstream sequence are evaluated. Data that used to establish this programming is derived from humans as well as from species other than human, since less is known at this point about SNP diversity in humans than about SNP diversity in lower organisms, including bacteria. As a result, the equations established are extrapolated for use in humans; their utility in aiding medical science to address needs related to finding antibiotics for resistant organisms remains valuable. Similarly drug resistant AIDS and cancers can be fruitfully investigated using this method. Examples of Quantitative Structure Activity Relationships (QSAR) for use in the described methods have been developed (7).

Another potential complication is the fact that the SNP in question may represent a step forward along the spectrum rather than backward. In this case, simplex methods or other similar methods can be used. Simplex algorithms allow for the projection into another plane of measurement from a known set of results for a multi-dimensional system.

### Measurements of Mutational Diversity

There are several types of matrices currently in use (and publically available) to measure mutational diversity. A catalog of the programs that use and incorporate these matrices is available from The European Bioinformatics Institute (EBI), a non-profit academic organization that is part of EMBL, the European Molecular Biology Laboratory. This catalog (Biocatalog) contains brief descriptions of over 500 publicly available algorithms. Website: http://www.ebi.ac.uk/biocat Text document. Programs include BLAST, PSI-BLAST, Smith-Waterman, Needleman-Wunsch, Hidden Markov Models, and FASTA8-13. Programs which use profiles, domains, and motifs are also available and contain measures which will be applied to this method (14-23).

Selected matrices are integrated into the NN or GA. Results will yield a conclusion that some of these measures are of little or no utility with SNPs whereas others are highly diagnostic for a particular disease state, or others are predictive of many disease states. SNPs that correlate to several disease states under this method will be of great interest as key reference points having a potential impact on several translational events or as yet undiscovered events. The DNA segments containing these SNPs, whether known genes or not, can then be prioritized targets that are highly likely to be involved in disease states and worthy of additional expenditure of resources to determine function.

Even more powerful matrices than those currently in existence are generated by applying the standard methods which were used to create these mutation matrices on the more subtle data which will arise from the studies made on SNPs, both collectively across all disease states and preferentially across one disease state. The choice of which matrices should be recomputed and the coefficients to be used to combine these matrices are guided by the results of the neural nets and genetic algorithms described above.

### Measurements of Protein Structural Diversity

As in the case of mutational matrices in use for bioinformatics, a series of measurements to describe the nature of changes in protein structure based on a single or multiple mutations is available. Much of this work is based on the physical changes as seen by x-ray crystallography and documented in the Protein DataBase maintained by the National Laboratories. This data is constantly growing and should grow at an even greater rate with the advent of several initiatives to establish high-throughput crystallography. These structures are used for virtual high-throughput screening applying force fields (24).

Other available methods include those of Eisenberg (25-27), Kabsch and Sander (28), Richardson (29,30), and an array of algorithms being developed in the field. CASP(31) is hosted by the Protein Structure Prediction Center, Biology and Biotechnology Research Program of Lawrence Livermore National Laboratory(32), and ExPASy (33, 34). Measurements of protein structural diversity as found in the algorithms judged to be the most useful in this competition are integrated into this method.

### Outcomes: Measurements of Disease State Severity or other Diagnostic Markers Measurements of Drug Therapy or other Treatment Response

In training a NN or GA, it is crucial to find a method of describing the results in a quantitative fashion. Some applications of the older QSAR methods in chemistry use only an outcome that says that the item being measured has activity or it does not. These methods are of limited utility and given the subtleties of using SNP data would be of only limited success. In the event that the quantitative outcomes described below are not available, then attempts may still be made to utilize otherwise useful data. In this case, GA's have historically been found to be of greater utility.

The methods described herein quantify the outcomes so that the scoring of the SNPs reflects the best possible predictive power of the data in hand. The methods include:

the stage of disease as found when the patient first reports to the doctor,

the degree of time lapsed between first recognition of the symptoms by the patient and the doctor visit

the rate of progress of the disease

the type of treatments given: dosage and timing

the degree of response by the patient as described by the doctor

the degree of response by the patient as described by the patient

genotyping data for the patient before, during, and after treatment

Some of these outcomes may be of limited use or the data unavailable. Others will form the needed information.

In the total absence of outcome information, it is not possible to use an NN or GA. In these cases, the measurements described above can still be used to formulate a probabilistic score based strictly on the measured values themselves using intelligent, manually constructed coefficients for the equations.

Using the claimed methods, it is possible to create a probabilistic score for any SNP of any given dataset. It is also possible to use that score to predict which SNPs are correlated to the disease and which are not. The DNA segments containing these highly predictive SNPs whether known genes or not are then presumed to be those that are most likely to be causative of disease rather than just effected by the disease state. These segments should then be the preferred target for drug discovery.

### Exemplary Applications

By extension, it is possible to create an overall SNP significance quotient where a particular SNP without medical data attached can be predicted to contain the potential for harm to the individual because of the type of damage that it can do, either by changing the protein structure or changing the body's response to the genetic or protein sequence.

The QSAR-SNP is used to predict another SNP that the individual should exhibit and then search that individual's genetic information to discover whether or not that individual has the currently known SNP distribution for that disease. If the individual does not then a new line of research is opened to discover the differences in the SNP distribution and their consequences on the patients disease and treatment progress.

Over time as the equations grow in diversity and applicability to the general population of humans or lower organisms, SNPs which have yet to be found either in the current population (for quickly growing organisms) or in the currently accepted nonjunk human DNA are predicted. One method of examining this possibility is the implementation of a reverse QSAR using standard QSAR methods coupled with energy calculations to predict next generation mutations in lower organisms that should reveal themselves as SNPs for that organism. This method is applicable to understanding antibiotic and anti-viral resistance as in the case MRSA (methicillin resistant S. aureus), VRE (vancomycin resistant enterococci), as well as drug resistance in AIDS and cancer. In the case of predicted SNPs in what has been termed “junk” DNA, DNA segments are re-examined with an eye to uncovering a previously unrecognized gene.

Measures of ADMET (absorption, distribution, metabolism, excretion, and toxicology) are incorporated in the methods described herein. The significance quotient of s SNP are modified according to a confidence value which will indicate its probabilistic participation in an ADMET condition. Targets with similar ADMET properties as based on this scoring method are grouped together to infer properties for targets not completely characterized. The methods allow evaluation of new methods of measurement and the software associated with them.

Another application is the coupling of this method with other types of medical information to enhance the prediction of the relative importance of targets. The method is coupled with competitive business information in order to obtain cross correlations between businesses that have targets with those that have drugs applicable to targets from the same family using the profiling techniques indicated above or similar methods.

SNPs contained in normal start codon sequences are exploited using this method. These SNPs can be expected to either render the translation machinery mute so that the protein is not expressed at all or create an acceleration or slow-down in the rate of production of the protein. These types of differential expression as seen in gene expression profiles receive a high priority score as they should be rich sources of drug discovery information about the individual's response to drugs and other treatments.

### Rone Biotechnology Index A

Part 1 of the index consists of a value used to connote the occurrence of a proline conversion (to or from) described below. Part 2 of the index correlates this information to known results for that type of conversion. Part 3 of the index consists of the attachment of a keyword(s) related to suspicions about the medical impact of the DNA segment, if any.

Part 4 of the index relates to information regarding the particular SNP when it is known. This part of the scoring connotes whether or not the residue under consideration either in DNA code or in protein code is a member of the binding site pocket, a controlling element in the binding, or the major partner in the binding, as found by experiment or as postulated by molecular modeling energy value considerations or homology to known proteins. Part 4 was not utilized in these preliminary results, but is expected to be a major contributing factor to the utility of the method.

Using the methods an index called Rone Biotechnology Index A was constructed to focus on conversions of DNA code from a proline amino acid residue to a non-proline residue and vice a versa. Previously it has been shown that proline conversions have a major effect on protein structures; even in some cases causing complete destruction of the activity of the protein. It is presumed therefore that a SNP coding for such a change has a very high priority index by our method. The implementation of the prototype is a manual search of public databases containing SNP information against the triplet code for proline recognizing that some DNA segments in the public domain may be forward or reverse reads of the code. Final implementation will be a fully automated system delivered via software programs. Results are described below.

Part 1 of the index used in the results are in simple binary format, 0 or 1. Either the proline conversion exists in the DNA segment examined or it does not. Modifications to this part of the index may incorporate:

the amount of structural damage anticipated from the conversion; conservative versus non-conservative substitution and other measures such as size and flexibility of side chains.

evidence of a known ORF which definitively signals a proline residue versus DNA segments of unknown ORF, the first having a higher weight in the method. Alternately this may be a separate index.

an overall adjustment index for this index relative to others that will be developed, if any is necessary.

### Application to Disease States

It has been established that female Down's syndrome patients have statistically fewer breast cancers than that of the normal population. When the population studies are adjusted for the shorter life span of Down's syndrome patients, the differences are less but still significant. Using this information, one can postulate that the extra copy of chromosome 21 confers some protective benefit. Consequently, we examined the public database concerning chromosome 21 was examined and searched for DNA segments which return a value of 1 on the Rone Biotechnology Index A, Part 1.

Examining WLAF segments on this chromosome the following segments have such a value:

WIAF-683: Pro to Leu* conversion

WIAF-1683: Pro to His* conversion

WIAF-2055: Pro to GLN* conversion

WIAF-508: Pro to GLN* conversion

WLAF-1500: Pro to Ser* conversion denotes a case where the ORF was not found during a examination of the literature obtainable on this DNA segment.

Using Parts 2 and 3 of the index, this information was correlated to known results for the types of conversion found and the suspected disease state “cancer”, adding a 1 to the score for each positive result. The case of WLAF-1500 returned a priority index then of 3 with the overall result that a similar conversion has been found to take place in the case of an enzyme (quinone oxireductase) which has been implicated in breast cancer.

The data indicate that these five segments are prioritized as targets for breast cancer drug therapies. Furthermore, in paradigm described herein, linking the three parts of the index, the data indicated that WIAF-1500 is the highest priority target.

### Extensions of Rone Index

The methods are used to formulate similar indices for all 20 amino acid residues (aar's) found in humans and for other aar's found in other organisms. Conversion indices may not be symmetrical in nature, i.e. a Proline to Leucine conversion will not have equal weight to a Leucine to Proline conversion. AAR's which are not part of the normal human makeup are selective targets for antiobiotics and antivirals and are be given higher scores when searching for preferential targets in these cases. Using these modified scores facilitates the search for sources of resistance to antibiotics and antivirals. Adjustments are made to look for SNPs associated with drug resistance in human cancers.

### Distinguishing Hallmarks and Advantages

One advantage of the methods is that it provides for combining measurements of mutational diversity with those of protein structural diversity into a single paradigm rather than using separate databases and disparate measurements. Another advantage is the formulation of relativistic measurements combining these two diversity measurements using a combination of neural nets, genetic algorithms, simplex methods, or specialized normalization techniques.

The construction of a knowledge-based expert system that reflects the reasoning of recognized experts in two different fields, biology and chemistry. The measurements for mutational diversity are used mostly in the field of molecular biology and bioinformatics and the measurements of protein structural diversity are used mainly in the field of molecular modeling and chemistry.

Other advantages include automation of the system so that it can be employed by the average laboratory scientist who is not an expert in these fields. Few scientists are experts in both fields. Even if they have knowledge of both and use the software packages commonly in use, they are still usually uneducated about the underlying operations of the measurements, the hidden assumptions embedded in the methods, and the proper use of one set of measurements versus another. This system provides them with simple way of prioritizing their targets and their drug/target combinations based on theoretical considerations which can then be verified in their laboratory.

Performance of an integrated system of measurements is higher than that of disparate systems, both in terms of CPU power required to perform the calculations and in terms of the quality of the results. If the case where neural nets and genetic algorithms are used, the system can be automated to train itself and define new QSAR-SNPs on an ad hoc basis for the laboratory scientist without requiring additional software coding resources or the input of an expert in the field. One of the current bottlenecks in the industry is the lack of personnel in either of these two fields who are highly trained in the internal workings of the computational techniques involved.

Combining measurements of mutational diversity with those of protein structural diversity into a single paradigm rather than using separate databases and disparate measurements.

Formulation of relativistic measurements combining these two diversity measurements using a combination of neural nets, genetic algorithms, simplex methods, or specialized normalization techniques.

The construction of a knowledge-based expert system that reflects the reasoning of recognized experts in two different fields, biology and chemistry. The measurements for mutational diversity are used mostly in the field of molecular biology and bioinformatics and the measurements of protein structural diversity are used mainly in the field of molecular modeling and chemistry.

The automation of this system so that it can be employed by the average laboratory scientist who is not an expert in these fields. Few scientists are experts in both fields. Even if they have knowledge of both and use the software packages commonly in use, they are still usually uneducated about the underlying operations of the measurements, the hidden assumptions embedded in the methods, and the proper use of one set of measurements versus another. This system will provide them with simple way of prioritizing their targets and their drug/target combinations based on theoretical considerations which can then be verified in their laboratory.

Performance of an integrated system of measurements will be higher than that of disparate systems, both in terms of CPU power required to perform the calculations and in terms of the quality of the results.

If the case where neural nets and genetic algorithms are used the system can be automated to train itself and define new QSAR-SNPs on an ad hoc basis for the laboratory scientist without requiring additional software coding resources or the input of an expert in the field. One of the current bottlenecks in the industry is the lack of personnel in either of these two fields who are highly trained in the internal workings of the computational techniques involved.

### Proline Conversion Index

In all cases, use of the term dna segment or genetic information does not preclude the conversion of all these methods into protein code elements (amino acid residues). The methods are fully interchangeable between the two types of data which may be available, dna code or protein code.

Part 1 of the index consists of a value used to connote the occurrence of a proline conversion (to or from) described below. Part 2 of the index correlates this information to known results for that type of conversion. Part 3 of the index consists of the attachment of a keyword(s) related to suspicions about the medical impact of the DNA segment, if any.

Part 4 of the index relates to information regarding the particular SNP when it is known. This part of the scoring connotes whether or not the residue under consideration either in DNA code or in protein code is a member of the binding site pocket, a controlling element in the binding, or the major partner in the binding, as found by experiment or as postulated by molecular modeling energy value considerations or homology to known proteins. Part 4 was not utilized in these preliminary results, but is expected to be a major contributing factor to the utility of the method.

Using our methods an index called Rone Biotechnology Index A was constructed to focus on conversions of DNA code from a proline amino acid residue to a non-proline residue and vice a versa. Previously it has been shown that proline conversions have a major effect on protein structures; even in some cases causing complete destruction of the activity of the protein. It is presumed therefore that a SNP coding for such a change should have a very high priority index by our method. The implementation of the prototype is a manual search of public databases containing SNP information against the triplet code for proline recognizing that some DNA segments in the public domain may be forward or reverse reads of the code. Final implementation will be a fully automated system delivered via software programs. Cited here are preliminary results.

Part 1 of the index used in the preliminary results are in simple binary format, 0 or 1. Either the proline conversion exists in the DNA segment examined or it does not. Future modifications to this part of the index will incorporate:

the amount of structural damage anticipated from the conversion; conservative versus non-conservative substitution and other measures such as size and flexibility of side chains.

evidence of a known ORF which definitively signals a proline residue versus DNA segments of unknown ORF, the first having a higher weight in the method. Alternately this may be a separate index.

an overall adjustment index for this index relative to others that will be developed, if any is necessary.

### Application

It has been established that female Down's syndrome patients have statistically fewer breast cancers than that of the normal population. When the population studies are adjusted for the shorter life span of Down's syndrome patients, the differences are less but still significant. Using this information one can postulate that the extra copy of chromosome 21 confers some protective benefit. Consequently we examined the public database concerning chromosome 21 and searching for DNA segments which return a value of 1 on the Rone Biotechnology Index A, Part 1.

Examining WIAF segments on this chromosome the following segments have such a value:

WIAF-683: Pro to Leu* conversion

WIAF-1683: Pro to His* conversion

WIAF-2055: Pro to GLN* conversion

WIAF-508: Pro to GLN* conversion

WIAF-1500: Pro to Ser* conversion denotes a case where the ORF was riot found during a examination of the literature obtainable on this DNA segment.

Using Parts 2 and 3 of the index we correlate this information to known results for the types of conversion found and the suspected disease state “cancer”, adding a 1 to the score for each positive result. The case of WIAF-1500 returns a priority index then of 3 with the overall result that a similar conversion has been found to take place in the case of an enzyme (quinone oxireductase) which has been implicated in breast cancer. The conclusion is that these five segments need to be more fully explored and understood with an eye to finding targets for breast cancer drug therapies. Furthermore, in our paradigm linking the three parts of the index we find that WIAF-1500 is the highest priority target by these methods.

### Extensions of this index

Similar indices are made for all 20 amino acid residues (aar's) found in humans and for other aar's found in other organisms. The conversion indices will not be symmetrical in nature, i.e. a Proline to Leucine conversion will not have equal weight to a Leucine to Proline conversion. AAR's which are not part of the normal human makeup are selective targets for antiobiotics and antivirals and will be given higher scores when searching for preferential targets in these cases. Using these modified scores, genes and mutation therein associated with resistance to antibiotics and antivirals are identified. Adjustments are made to look for SNPs associated with drug resistance in human cancers.

### EXAMPLE

Genes on human chromosomes 10-22 were analyzed and catalogued with probabalistic scores indicative of disease correlations. The following definition files and chromosome catalogues were produced.

Definition file containing the definition of the Rone Biotechnology Index A, Part 1 in electronic form suitable for loading into documents or software to evaluate SNPs probabilistic scoring

Catalog of SNPs from Chromosome 21 containing a priority rating of 1 for Rone Biotechnology Index A, Part 1.

Definition file containing the definition of the Rone Biotechnology Index A, Part 1 in electronic form suitable for loading into documents or software to evaluate SNPs for probabilistic scoring.

Definition file containing the definition of the Rone Biotechnology Index B, Part 1 in electronic form suitable for loading into documents or software to evaluate SNPs for probabilistic scoring.

Catalog of SNPs from Chromosome 10 containing a priority rating of 1 for Rone Biotechnology Index A, Part 1. This represents 3219 SNPs out of 50,907 as high priority (6.3%).

Catalog of SNPs from Chromosome 11 containing a priority rating of 1 for Rone Biotechnology Index A, Part 1. This represents 3196 SNPs out of 52,046 as high priority (6.1%).

Catalog of SNPs from Chromosome 12 containing a priority rating of 1 for Rone Biotechnology Index A, Part 1. This represents 3152 SNPs out of 45208 as high priority (7.0%).

Catalog of SNPs from Chromosome 13 containing a priority rating of 1 for Rone Biotechnology Index A, Part 1. This represents 2805 SNPs out of 43,223 as high priority (6.5%).

Catalog of SNPs from Chromosome 14 containing a priority rating of 1 for Rone Biotechnology Index A, Part 1. This represents 3002 SNPs out of 38,581 as high priority (7.8%).

Catalog of SNPs from Chromosome 15 containing a priority rating of 1 for Rone Biotechnology Index A, Part 1. This represents 3178 SNPs out of 31,670 as high priority (10.0%).

Catalog of SNPs from Chromosome 16 containing a priority rating of 1 for Rone Biotechnology Index A, Part 1. This represents 3323 SNPs out of 29,522 as high priority (11.3%).

Catalog of SNPs from Chromosome 17 containing a priority rating of 1 for Rone Biotechnology Index A, Part 1. This represents 3392 SNPs out of 23,922 as high priority (14.2%).

Catalog of SNPs from Chromosome 18 containing a priority rating of 1 for Rone Biotechnology Index A, Part 1. This represents 2909 SNPs out of 30,495 as high priority (9.5%).

Catalog of SNPs from Chromosome 19 containing a priority rating of 1 for Rone Biotechnology Index A, Part 1. This represents 3559 SNPs out of 16,559 as high priority (21.5%).

Catalog of SNPs from Chromosome 20 containing a priority rating of 1 for Rone Biotechnology Index A, Part 1. This represents 3346 SNPs out of 24,032 as high priority (13.9%).

Catalog of SNPs from Chromosome 21 containing a priority rating of 1 for Rone Biotechnology Index A, Part 1. This represents 2885 SNPs out of 16,391 as high priority (17.6%).

Catalog of SNPs from Chromosome 22 containing a priority rating of 1 for Rone Biotechnology Index A, Part 1. This represents 3619 SNPs out of 17,210 as high priority (21.0%).

Catalog of SNPs from Chromosome Y containing a priority rating of 1 for Rone Biotechnology Index A, Part 1. This represents 102 SNPs out of 586 as high priority (17.4%).

Table 1 shows a summary of the data derived using the probabalistic scoring method described herein. A “high priority” SNP indicates an increased probability of disease-correlation.

Table 1

| | | | |
|---|---|---|---|
| | # of High | | Percentage of High |
| Chromosome | Priority SNPs | # of Total SNPs | Priority SNPs |

| | | | |
|---|---|---|---|
| 10 | 3219 | 50,907 | 6.3% |
| 11 | 3196 | 52,046 | 6.1% |
| 12 | 3152 | 45208 | 7.0% |
| 13 | 2805 | 43,223 | 6.5% |
| 14 | 3002 | 38,581 | 7.8% |
| 15 | 3178 | 31,670 | 10.0% |
| 16 | 3323 | 29,522 | 11.3% |
| 17 | 3392 | 23,922 | 14.2% |
| 18 | 2909 | 30,495 | 9.5% |
| 19 | 3559 | 16,559 | 21.5% |
| 20 | 3346 | 24,032 | 13.9% |
| 21 | 2885 | 16,391 | 17.6% |
| 22 | 3619 | 17,210 | 21.0% |
| Y | 102 | 586 | 17.4% |

### Reference

1. Drysdale C, et al. “Complex Promoter and Coding Region of beta ₂-adrenergic Receptor Haplotypes Alter Receptor Expression and Predict In Vivo Responsiveness,” Proceedings of the National Academy of Sciences (tiNAS). 2000; 97:483-10488.

2. Clark, A. G (1990) Mol. Biol. Evol. 7, 111-122.

3. Excoffier, L., Smouse, P. E. & Quattro, J. M. (1992) Genetics 131, 479-491.

4. Hill, W. G. & Robertson, A. (1968) Theor. Appl. Genet. 38, 226-231.

5. Hill, W. G. & Weir, B. S. (1994) Am. J. Hum. Genet. 54, 705-714.

6. Ludbrok, J. (1998) Clin. Exp. Pharmacol. Physiol. 25, 1032-1037.

7. Walter, D. E., and Hinds, R. M., “Genetically Evolved Receptor Models (GERM): A Computational Approach to Construction of Receptor Models”, J. Med. Chem. 37,2527 (994)

8. BLAST and its modifications, including PSI-BLAST: Altschul S. F. and Gish W.; “Local alignment statistics.”; Methods in Enzymology 266:460-80(1996). http;//blast. wustl.edu

9. CLUSTALW: Thompson J. D. et al. “CLUSTAL W: improving the sensitivity of progressive multiple sequence alignment through sequence weighting, position-specific gap penalties and weight matrix choice.” Nucleic Acid Research. 1994;22:4673-4680.

10. FASTA: Lipman D. J. and Pearson W. R. Science 1985; 227: 1435-1441

11. HMM: Sean Eddy et al at Washington University, Saint Louis (http://hmmer.wustl.edu/).

12. Needleman S and Wunsch C. “A general method applicable to the search for similarities in the amino acid sequences of two proteins.” Journal of Molecular Biology. 1970; 48:444-453.

13. Smith T and Waterman M S. Identification of common molecular subsequences. Journal of Molecular Biology. 1981; 147:195-197.

14. Gribskov M et al. “Profile Analysis.” Methods in Enzymology. 1987;183:146-159.

15. Feng D and Doolittle R. F. “Progressive sequence alignment as a prerequisite to correct phylogenetic trees.” Journal of Molecular Evolution. 1987;25:351-360.

16. Hofmann K et al. “The PROSITE database, its status in 1999.” Nucleic Acids Research. 1999;27:215-219.

17. InterPro at http://www.ebi.ac.uk/˜wfl/intertalkismb/)

18. BLOCKS (http://www.blocks.fhcrc.org/).

19. The PRINTS website: [symbol] http://bioinf.man.ac.uk/dbbrowser/PRINTS/

20. The ProDom database (http://protein.toulouse.inra.fr/prodom.html)

21. Pfam from the Sanger Centre (http://www.sanger.ac.uk/Software/Pfam/).

22. Phrap program by Phil Green of the University of Washington.

23. Worley, K. C., “BEAUTY: An enhanced BLAST-based search tool that integrates multiple biological information resources into sequence similarity search results.”; Genome Research 5:173-184 (1995).

24. F. A. Momany and R. Rone, “Validation of the General Purpose QUANTA 3.2/CHARMm Force Field,” J. Comp. Chem. 13 (7), 888-900 (1992).

25. Eisenberg D., Marcotte E. M., Xenarios I., Yeates T. O. Protein function in the post-genomic era. Nature 405, 823-826 (2000).

26. Localizing proteins in the cell from their phylogenetic profiles. Marcotte E. M, Xenarios I, van Der Bliek A. M, Eisenberg D. Proc Natl Acad Sci U S A. Oct. 24, 2000 97(22): 12115-20.

27. Selecting protein targets for structural genomics of Pyrobaculum aerophilum: validating automated fold assignment methods by using binary hypothesis testing. P. Mallick, K. E. Goodwill, S. Fitz-Gibbon, J. H. Miller, D. Eisenberg Proc Natl Acad Sci U S A 97(6): 2450-5 (2000).

28. W. Kabsch and C. Sander, Dictionary of Protein Secondary Structure: Pattern Recognition of Hydrogen-Bonded and Geometrical Features, Biopolymers, 22, 2577 (1983)

29. Jane S. Richardson, The Anatomy and Taxonomy of Protein Structure, Advances in Protein Chemistry, 34, 167-218 (1981).

30. Richardson, J. S., Getzoff, D. C., and Richardson, D. C., Proceedings of the National Academy of Science, USA 75, 2574-2578 (1978).

31. CASP: http://predictioncenter.llnl.gov/casp4/Casp4.html

32. Protein Structure Prediction Center, Biology and Biotechnology Research Program of Lawrence Livermore National Laboratory: http://predictioncenter.llnl.gov/

33. ExPASy Molecular Biology Server http://www.expasy.ch/

34. ExPASy Protein Structure tools: http://www.expasy.ch/tools/#primary What is claimed is:

1. A method of identifying a disease-associated gene, the method comprising:
obtaining first profiles of genes that are free of SNPs and corresponding second profiles of the genes containing at least one SNP;
for each SNP residue in the second profiles, analyzing indicia of triplets of residues in the second profiles containing the SNP residue and corresponding triplets in the first profiles to determine whether any of the corresponding triplets indicate a proline conversion between the first and second profiles;
adding indicia of proline conversions for each of the genes to determine probability scores to identify a disease-associated gene.

2. The method of claim 1 further comprising altering the probability scores in accordance with ontological information associated with the genes.

3. A chromosome profile comprising a catalog of SNPs, wherein a SNP is prioritized with a probabalistic score.
